(19) United States
(12) Patent Application Publication
(10) Pub. No.: US 2006/0129151 A1
(43) Pub. Date: Jun. 15, 2006

(54) Systems and methods for securing fractures using plates and cable clamps

(76) Inventors: C. Wayne Allen, Southaven, MS (US); Darin Gerlach, Cordova, TN (US)

Correspondence Address: CHIEF PATENT COUNSEL;SMITH & NEPHEW, INC., 1450 BROOKS ROAD, MEMPHIS, TN, 38116, US

(21) Appl. No.: 11/259,854

(22) Filed: Oct. 26, 2005

Related U.S. Application Data
(63) Continuation-in-part of application No. 10/952,047, filed on Sep. 28, 2004.
Application No. 10/952,047 is a continuation-in-part of application No. 10/673,833, filed on Sep. 29, 2003.
(63) Continuation of application No. 10/230,040, filed on Aug. 28, 2002.

(51) Int. Cl. A61F 2/30

(52) U.S. Cl. 606/69

(57) ABSTRACT

Systems, devices, and methods of using bone plates with cable clamps to help secure bone plates in the desired positions for securing and treating bone fractures. The bone plates are adapted to be used interchangeably with bone screws, compression screws, or cable clamps.

This application is a continuation-in-part of U.S. patent application Ser. No. 10/952,047 filed Sep. 28, 2004 titled “Bone Plates and Methods for Provisional Fixation Using Same” which is a continuation-in-part of U.S. application Ser. No. 10/673,833, filed on Sep. 29, 2003. This application is also a continuation of U.S. patent application Ser. No. 10/230,040 filed Aug. 28, 2002 titled “Systems, Methods and Apparatuses for Clamping and Re-Clamping an Orthopedic Surgical Cable.” The entire contents of each of the above-identified patent applications are hereby incorporated by reference.

### FIELD OF THE INVENTION

The present invention relates to systems, devices, and methods of using bone plates with cable clamps to help secure bone plates in the desired positions for securing and treating bone fractures.

### BACKGROUND

Bone fractures often lead to complex tissue injuries involving both the bone and the surrounding soft tissue. Treated in a conservative way, fractures often result in malalignment or non-unions and may also lead to stiffness of adjacent joints. Open reduction and internal fixation of the bone can reduce the occurrence of these problems. Anatomical reduction and stable internal fixation with plates and screws are also quite successful in treating bone fractures.

Good bone healing can also result from relative stability, where the clinical outcome is often dependent upon obtaining correct length, axis, and rotation of the fractured bone rather than upon precise anatomical reduction and absolute stability. To achieve stability, while at the same time minimizing the amount of additional soft tissue trauma, treatment of multi-fragmented metaphyseal and diaphyseal fractures with plates and screws is often used.

In some instances, a bone screw is threaded into bone and used to compress the bone against the plate. This solution often results in high strain that can loosen the plate and screw from the bone.

One example of a solution to that problem, however, is a plate and screw system that has the screws locked into the plate. The plate and screws form one stable system, the stability of the fracture is dependent upon the stiffness of the construct, and the angular relationship between the plate and the screw is maintained. No compression of the plate onto the bone is required, which reduces the risk of loss of reduction and preserves bone blood supply. Locking the screw into the plate to ensure angular, as well as axial, stability eliminates the possibility for the screw to toggle, slide, or be dislodged, and thereby reduces the risk of postoperative loss of reduction. Because the relationship between the locking screw (or screws) and the plate is fixed, locking screws provide a high resistance to shear or torsional forces. However, locking screws have a limited capability to compress bone fragments.

Existing plates with openings that accept locking screws, typically only accept certain screw sizes with specified types of screw heads. This may cause challenges because certain surgeries require the use of a lag screw with a shallow threadform and a conical screw head. Existing plates that accept only locking screws limit the angulation of a lag screw. This may be limiting in certain cases, for example with a distal femur fracture where a surgeon desires to lag the condyles. Because such existing plates do not accept lag screws with spherical screw heads, surgeons are limited to lagging fragments outside the plate or using screws that are poorly designed for this application.

Because of these shortcomings, it is desirable to provide plate and screw systems or bone plate assemblies that allow the surgeon to choose intraoperatively whether to use the bone plate with compression or lag fixation elements or screws (also referred to as non-locking screws), locking fixation elements or screws, or with a combination of both. In an effort to meet this desire, some plates provide a combination slot, which is a compression slot combined with a partially threaded opening that can receive either a compression screw or a locking screw. The partially threaded portions allow either locking or compression screws to be used. However, because the slots are only partially threaded, the locking screws may not be able to maintain the fixed angular relationship between the screws and plate under physiological loads. Specifically, the locking screws within the plate are only partially captured and thus only partially surrounded by threads. Under high stress and loading conditions, the slot may distort and allow the fixed angular relationship between the locking screw and plate to change. This can result in loss of fixation or loss of established intraoperative plate orientation. Additionally, because of the slot geometry of the combination slots, translation of the plate with compression screws may be limited to a single direction, which may be disadvantageous in reduction and manipulation of fragments.

Accordingly, there is a need for improved bone plates that may be used with both compression and locking fixation elements or screws for improved stabilization and compression of parts of a fractured bone. There is also a need for improved bone plates with holes that may be used for locking a bone plate to the bone, but that also accept different size fixation elements with varying types of heads.

Moreover, as the surgeon is preparing to reduce the fracture with a bone plate, he or she may wish to additionally use a surgical cable to secure the bone plate into place. This could be in addition to, or in place of, the above-described screws and other fixation elements. For example, in an orthopedic surgical procedure, surgically implanted orthopedic cables are frequently used to secure bones together, or otherwise used to tie or fit other parts of the body together. An orthopedic cable is typically a thin length of cable that is manufactured from a biocompatible material such as cobalt chromium alloy, or stainless steel, or another similar type of material. Generally, an orthopedic cable is wrapped around an affected area of a patient's bone structure and then secured with a device, such as a cable crimping device, in order to stabilize the bone, secure fractures, stabilize trauma, install other devices to the bone, and for other purposes. Conventional orthopedic cable products use a cable crimping device to crimp the orthopedic cable and secure the cable with a specific tension around the affected area of a patient's body.

However, crimping the cable typically causes damage to the cable and renders it unsuitable for re-use in an orthopedic procedure. It is not uncommon for an orthopedic cable to be replaced during the same surgical procedure when the tension on the orthopedic cable is insufficient and the cable must be retightened to obtain a sufficient tension. Because the orthopedic cable is often damaged due to the crimping procedure, it must be replaced. In other words, each time an orthopedic cable is tensioned with respect to the patient's femur, the bone plate becomes further secured to the exterior of the patient's femur. However, as each orthopedic cable is tensioned, other previously tensioned orthopedic cables may loosen, or the position of the orthopedic device may shift. In either instance, previously tensioned orthopedic cables may have to be re-tensioned or re-positioned with respect to the bone plate and the patient's femur. Conventional orthopedic cable products or devices used to secure the position of the orthopedic cables may have to be replaced along with the orthopedic cables that have become damaged or crushed due to the installation of the orthopedic cable products or devices. Replacing the orthopedic cable during a surgical procedure is time consuming for the surgeon and increases the cost of the surgery because the original orthopedic cable has been wasted.

In some instances, the conventional orthopedic cable product or portions of the product must also be replaced. In order to save time, manufacturers have designed single-use devices to secure the position of an orthopedic cable in a patient's body. These single-use devices cannot be reused and must be discarded if the orthopedic cable is initially tensioned and changes in the tension or position of the surgical cable must be made later. For example, one conventional orthopedic cable product uses a deformable sleeve or tube around the orthopedic cable. The metal sleeve or tube is then deformed by a screw that compresses the parts of the sleeve or tube around the cable. Once used, the sleeve or tube is deformed or crushed, and cannot be reused. The orthopedic cable may also become deformed or crushed and unsuitable for re-use. In either event, once the surgical cable has been set to a desired position or tension, and for any reason it becomes necessary to re-position or re-tension the surgical cable, the sleeve or tube or the conventional orthopedic cable product must be replaced.

At least one conventional orthopedic cable product uses a releasable lever-operated cable clamp to apply a clamping force to an orthopedic cable. The conventional orthopedic cable product tensions the cable to a desired tension, and a crimp is swaged onto the cable to hold the tension. Then the lever-operated cable clamp releases the clamping force, and the cable clamp is removed from the cable. This type of orthopedic cable product is not implantable within a patient's body because the lever-operated cable clamp is a separate component from the crimp, and it is too large for implanting in a body. Products featuring a non-implantable clamp add to the complexity and time for performing surgical procedures.

Accordingly, the present inventions seeks to provide improved and versatile bone plates that can be secured in a myriad of ways, e.g., by locking fixation elements or screws, by non-locking fixation elements or screws, by other fixation elements, and by cable and clamp combinations.

### SUMMARY

Various embodiments include a bone plate assembly that comprises a bone plate (or other orthopedic device) adapted to receive at least one locking element, at least one compression element, an optional provisional fixation pin, and at least one cable clamp incorporated into the bone plate. The bone plate includes an upper surface, a bone contacting surface, and a plurality of holes for receiving bone fixation elements, wherein each hole extends through the upper surface and the bone contacting surface, may interchangeably receive a locking fixation element and/or a compression fixation element (or any other fixation element), and includes a thread that makes a complete revolution around the hole. An optional provisional fixation pin may be received Within a smaller pinnacle or at least one of the plurality of holes for receiving bone screws. As used in this document, “screw” or “fixation element” may mean any fixation element that has any type of thread thereon or any other element intended to be used to treat or repair bone fractures. The threads need not have a particular pitch or shape, and “screw” encompasses all components that are intended to engage bone to another element per treatment, and includes fixation elements having blades, moly bolts, ridges, talons, locking pegs, or any other structure.

The preferred bone plate also has a surgical cable clamp incorporated into the bone plate for clamping and reclamping an orthopedic surgical cable. The clamp features a clamping mechanism that includes a clamping body and a securing member. The cable clamp is designed so that it preferably does not damage the orthopedic surgical cable when then the clamp is operated or clamped with respect to the surgical cable. While the surgical cable is operated or in use, a tension can be maintained on the orthopedic surgical cable. Furthermore, the surgical cable clamp can be reused along with the same surgical cable when the surgical cable clamp is unclamped and reclamped with respect to the surgical cable, while retensioning the surgical cable with respect to the bone plate. Such systems, methods, and apparatuses are particularly useful for surgeons installing an orthopedic surgical cable within a patient's body, and attempting to tension and retension the orthopedic cable with respect to the installation of the bone plate in the patient's body, while also securing the bone plate with locking or non-locking fixation elements. The bone plate and cable clamp combination may also be provided with a series of fixation structures and surgical cables for use.

### BRIEF DESCRIPTION OF THE DRAWINGS

FIG. 1 shows an exemplary bone plate according to one embodiment of the invention.

FIG. 2 shows another bone plate having cable clamps incorporated into the plate.

FIG. 3A shows a side view of an exemplary locking fixation element.

FIG. 3B shows a cross-sectional view of the fixation element and FIG. 3A.

FIG. 4A shows a top view of a portion of a bone plate having a hole without the threads shown.

FIG. 4B shows a cross-sectional view of the bone plate of FIG. 4A as viewed along the lines 4B.

FIG. 4C shows a top portion of the bone plate of FIG. 4A with the threads of the hole shown.

FIG. 4D shows a side view of a locking fixation element threaded into the portion of the bone plate shown in FIGS. 4A-4C, as viewed along the lines 4D in FIG. 4C.

FIG. 5A shows a side view of an exemplary compression fixation element.

FIG. 5B shows a side view of the compression fixation element of FIG. 5A inserted into the bone plate shown in FIGS. 4A-4C.

FIG. 6A shows a side view of another exemplary locking fixation element that may be used with embodiments of this invention.

FIG. 6B shows a cross-sectional view of the locking fixation element of FIG. 6A.

FIG. 7A shows a top view of a portion of a bone plate according to other embodiments of this invention.

FIG. 7B shows a cross-sectional view of the bone plate of FIG. 7A along the lines 7B.

FIG. 7C shows a detailed view of the hole of the bone plate of FIGS. 7A and 7B.

FIG. 8 shows a side view of the locking fixation element of FIGS. 6A and 6B inserted into the bone plate of FIGS. 7A-7C.

FIG. 9 shows a side view of the compression fixation element of FIG. 5A inserted into the bone plate of FIGS. 7A-7C.

FIG. 10 shows a side view of a portion of a bone plate having a clamping mechanism associated therewith.

FIG. 11 shows a cross-sectional view of another embodiment of a bone plate and clamping mechanism.

FIGS. 12-13 show cross-sectional views of further embodiments of a bone plate and clamping mechanism.

FIG. 14 shows an alternate embodiment of a bone plate having a wing nut clamping mechanism.

FIGS. 15A and 15B show side views of a clamping mechanism that has a hinge.

FIG. 16 shows a side view of a clamping mechanism that has a spring.

### DETAILED DESCRIPTION OF THE DRAWINGS

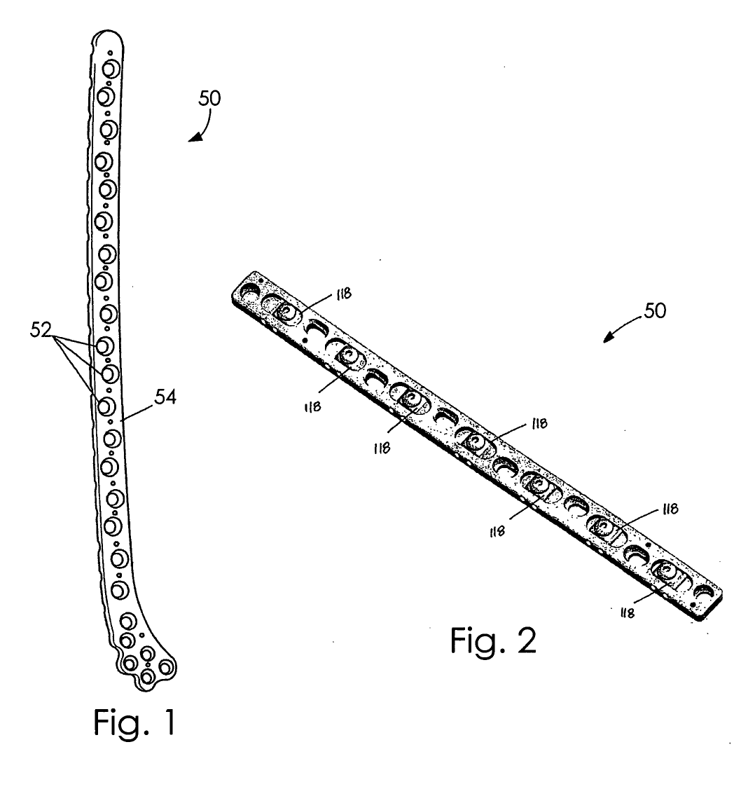

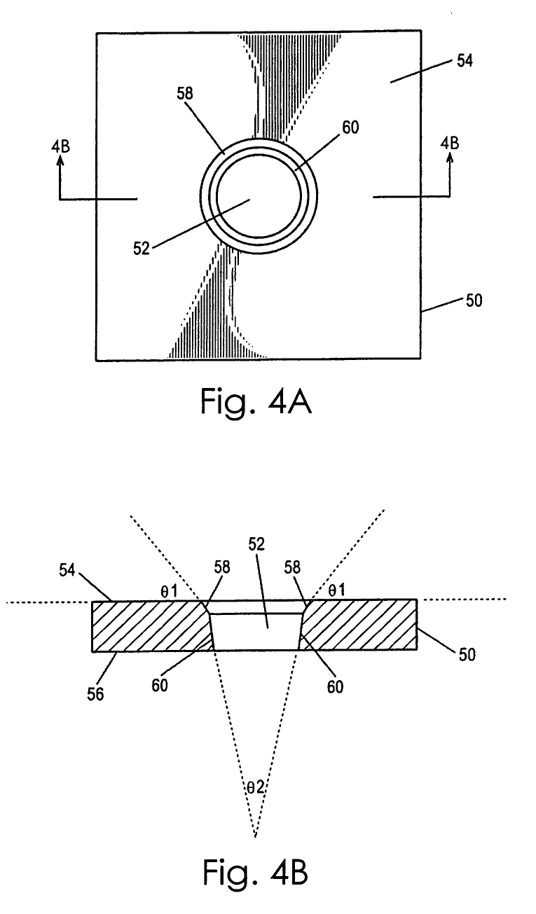

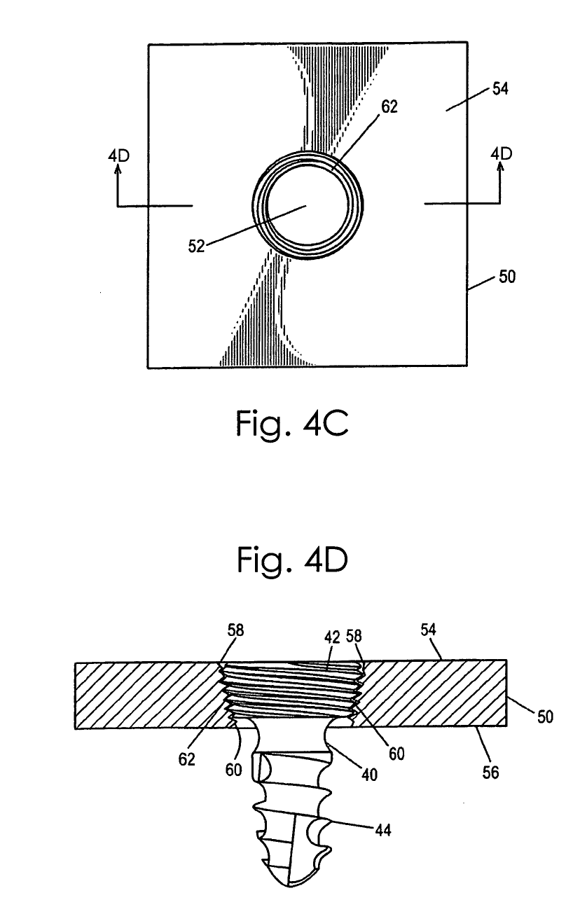

Embodiments of the present invention provide bone plate assemblies with cable clamps for stabilization and compression of parts of a fractured bone. As shown in FIG. 1, a bone plate 50 includes an upper surface 54, a bone contacting surface 56 (shown in FIG. 4B), and at least one hole 52 extending between the upper surface 54 and the bone contacting surface 56 that may interchangeably receive a locking fixation element or screw and/or a compression fixation element or screw, described more below. Each hole 52 preferably has a thread 62 (shown in FIGS. 4C and 4D) that makes at least one complete revolution around the hole 52. The threads of each hole may be configured to receive threads of a head of a locking screw. Each hole may be configured to threadably engage a head of a locking screw and fix the locking screw with respect to the bone plate. Each hole may also be configured to engage a head of a compression screw and provide compression of fractured bone fragments.

In some embodiments, a threaded head of a locking screw for use in accordance with this invention is received by threads in a corresponding hole such that the threads of the hole completely surround the threads of the head of the locking screw. This relationship between the head of the locking screw and the threads of the hole contributes to maintaining fixation of the bone plate and strengthening the plate and screw combination. As noted, and as will be described in more detail below, a compression screw may also be received within the hole of the bone plate. As the compression screw is fully inserted within a bone, the head of the compression screw comes into contact with and rides along a top portion of the hole, allowing for fine adjustment of the position of the bone plate in more than one direction.

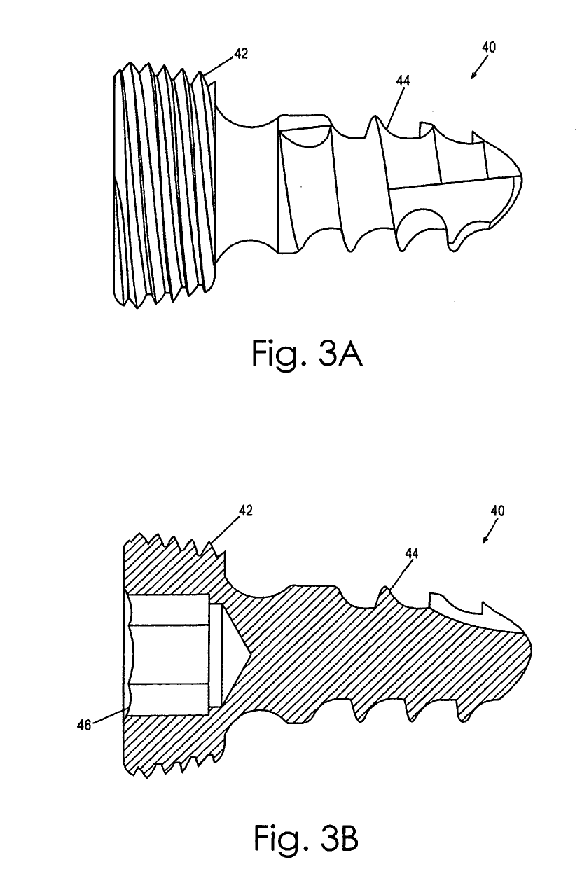

FIGS. 3A and 3B show an exemplary locking screw 40 for use according to one embodiment of the present invention. A locking screw 40 includes a threaded head 42 and a threaded shaft 44. Locking screw 40 may be a 3.5 mm, 4.5 mm, 6.5 mm, or other size locking screw. In the example shown, the lead between the threads of head 42 and the threads of shaft 44 is broken. In the embodiment shown, the threads in shaft 44 of locking screw 40 are single lead and the threads in head 42 are triple lead, providing locking screw 40 with same pitch throughout. It is preferable for certain embodiments of locking screws according to this invention to have a constant pitch, however, it should be understood that any number of leads and pitches may be provided and used accordingly with the bone plates described herein. As shown in FIG. 3B, locking screw 40 also includes an internal hex head 46 that is used when tightening locking screw 40 into a bone plate and/or bone.

FIGS. 4A-4C show various views of holes 52 in bone plate 50. For ease of illustration and for purposes of describing an exemplary embodiment of the present invention, only a portion of bone plate 50 is shown in FIGS. 4A-4C. Bone plates generally include one or more holes 52 or other openings (which may include pinholes or provisional fixation holes that cannot receive bone screws, briefly described below). Although not shown, in addition to holes that may receive either locking screws or compression screws interchangeably, bone plates 50 may also include other holes, for example, other oblong or non-threaded openings for receiving bone screws.

The bone plate portion 50 shown in FIGS. 4A-4C includes a hole 52 extending through an upper surface 54 and a bone contacting surface 56. FIG. 4A shows hole 52 without its threads to help illustrate certain aspects of this embodiment of the invention, while FIG. 4C shows hole 52 with its thread 62. FIG. 4B shows hole 52 having an upper portion 58 extending downward from upper surface 54. Upper portion 58 extends from upper surface 54 at an angle of θ1 relative to the plane of upper surface 54. In an exemplary embodiment, angle θ1 is about fifty-two degrees.

A bottom portion 60 of hole 52 extends from the end of upper portion 58 through bone contacting surface 56 of bone plate 50. Bottom portion 60 includes threads 62, as shown in FIG. 4C. Some of threads 62 may extend into upper portion 58 depending on the particular embodiment, but upper portion 58 need not be, and may not be, completely threaded.

Bottom portion 60 is shown tapered. The included angle θ2 shown in FIG. 4B, of the taper of bottom portion 60 may be less than about thirty degrees, including zero degrees (i.e., no taper at all). The larger the included angle, the larger hole 52 in bone plate 50 must be, which begins to compromise the strength of the plate if the included angle is much larger than about thirty degrees. In an exemplary embodiment, θ2 is about twenty degrees.

FIG. 4D shows a side view of locking screw 40 threaded into hole 52 of bone plate 50. Head 42 of locking screw 40 is received by threads 62 of bone plate 50. Threads 62 completely surround the threads of head 42, and the top of head 42 is received completely within hole 52 such that head 42 of locking screw 40 preferably sits flush with upper surface 54 of bone plate 50. Shaft 44 of locking screw 40 is threaded into bone (not shown). Head 42 of locking screw 40 is preferably tapered such that it properly mates with threads 62 of hole 52 of bone plate 50. Furthermore, a threaded portion of a head of a locking screw for use with certain embodiments of this invention should preferably have a taper generally corresponding to the taper, if any, of the threads of the hole of the bone plate.

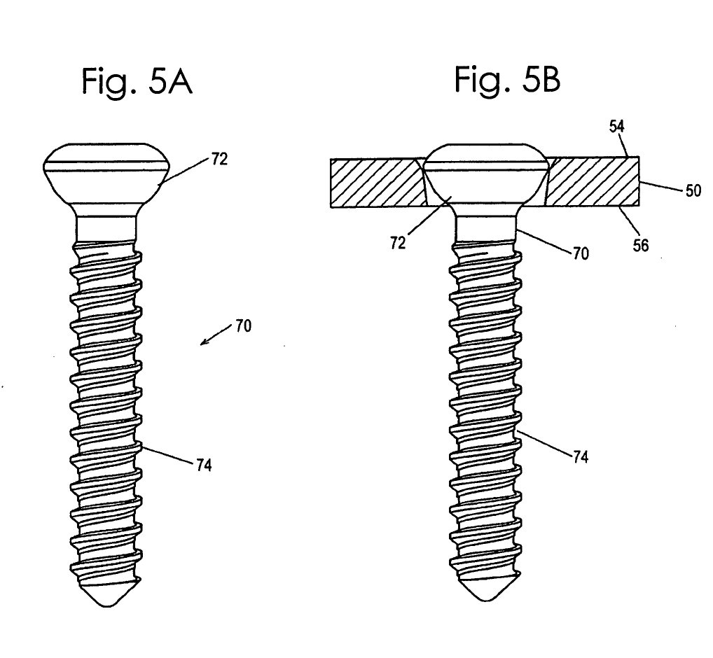

FIG. 5A shows a side view of an exemplary compression screw 70 for use according to an embodiment of the present invention. A compression screw 70 includes a head 72 and a threaded shaft 74. FIG. 5B shows compression screw 70 inserted within hole 52 of bone plate 50. As shown in FIG. 5B, head 72 of compression screw 70 rides along upper portion 58 of bone plate 50. As shaft 74 is threaded into a bone (not shown), compression screw 70 may pull or push bone plate 50 in a particular direction as head 72 of compression screw 70 comes into contact with and rides along upper portion 58 of hole 52. The angle θ1 at upper portion 58 of hole 52 is significant for compression of a fracture and is necessary to help shift the bone plate in the desired direction. (If upper portion 58 were to extend straight down from upper surface 54 of bone plate 50, it is likely that compression would be less successful.) Compression screw 70 may move bone plate 50 in more than one direction as compression screw 70 is fully inserted within hole 52. In an exemplary embodiment, fine adjustment of fractures up to about two millimeters in several directions is possible.

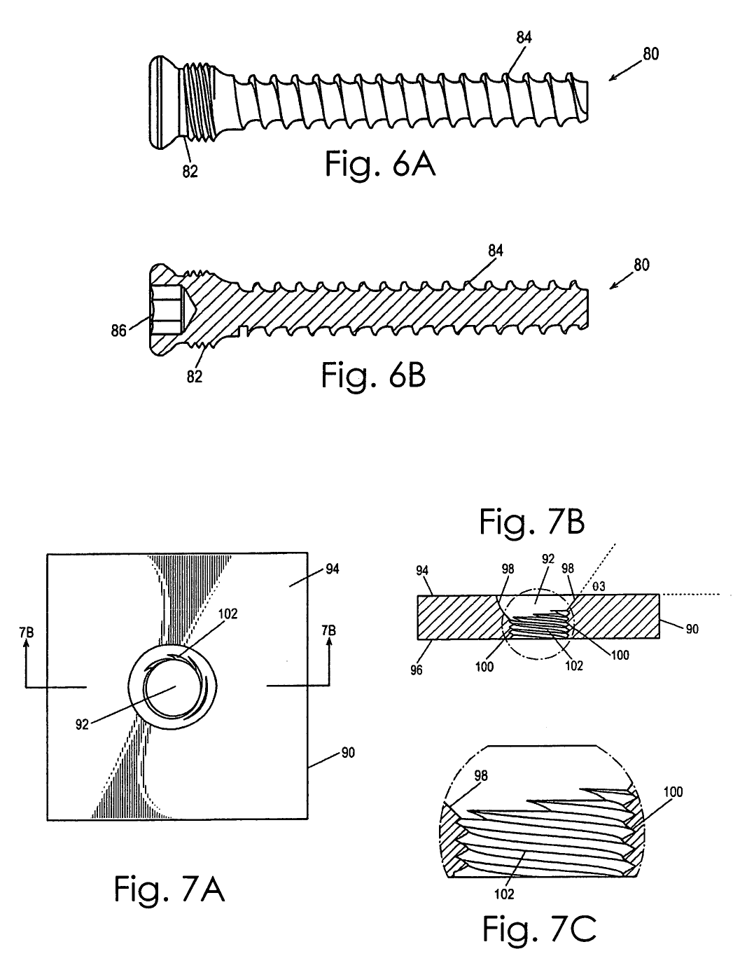

FIGS. 6A and 6B show another exemplary locking screw for use according to an embodiment of the present invention. A locking screw 80 includes a head 82 and a threaded shaft 84. Similar to locking screw 40 shown in FIGS. 3A and 3B, locking screw 80 may be a 3.5 mm, 4.5 mm, 6.5 mm, or other size locking screw, and the lead between the threads of head 82 and the threads of shaft 84 may be broken. The threads in shaft 84 of locking screw 80 are preferably single lead and the threads in head 82 are triple lead, providing locking screw 80 with the same pitch throughout. The pitches and angles of thread form for exemplary 3.5 and 4.5 mm locking screws 80 are generally similar to those described above with reference to locking screw 40.

Locking screw 80 also includes an internal hex head 86, as shown in FIG. 6B, that is used when tightening locking screw 80 into a bone plate and/or bone. As may be seen from FIGS. 3A, 3B, 6A, and 6B, only a portion of head 82 of locking screw 80 is threaded, whereas the entire head 42 of locking screw 40 is threaded. Additionally, the threaded portion of head 82 of locking screw 80 is preferably not tapered, whereas head 42 of locking screw 40 is tapered. In the preferred embodiment, locking screw 40 is designed to mate with hole 52 of bone plate 50, while locking screw 80 is designed to mate with a hole 92 of a bone plate 90, as further described below.

FIGS. 7A-7C show different views of a portion of a bone plate 90 according to an alternate embodiment. These features, along with the features described above, may be incorporated into a single bone plate if desired. As noted above, bone plates generally include one or more holes or other openings. As shown in FIG. 7A, bone plate 90 portion includes a hole 92 extending through an upper surface 94 and a bone contacting surface 96 of bone plate 90. Hole 92 includes a top portion 98 extending downward from upper surface 94. As shown in FIG. 7B, one side of top portion 98 includes a ramp that extends from upper surface 94 at an angle of θ3 relative to the plane of top surface 94. In an exemplary embodiment, angle θ3 is about fifty-two degrees. The remainder of top portion 98 is a concave recessed portion that is generally spherical in shape, as shown in FIG. 7C.

A bottom portion 100 of hole 92 extends from the end of top portion 98 through bone contacting surface 96 of bone plate 90. Bottom portion 100 includes threads 102. Some of threads 102 may extend into top portion 98 depending on the particular embodiment, but top portion 98 preferably generally has only the beginning of thread leads, if any threading. In one embodiment, bottom portion 100 is not tapered, but rather is generally cylindrical in shape. In certain embodiments, for example, bottom portion 100 may be tapered at an included angle of less than about thirty degrees.

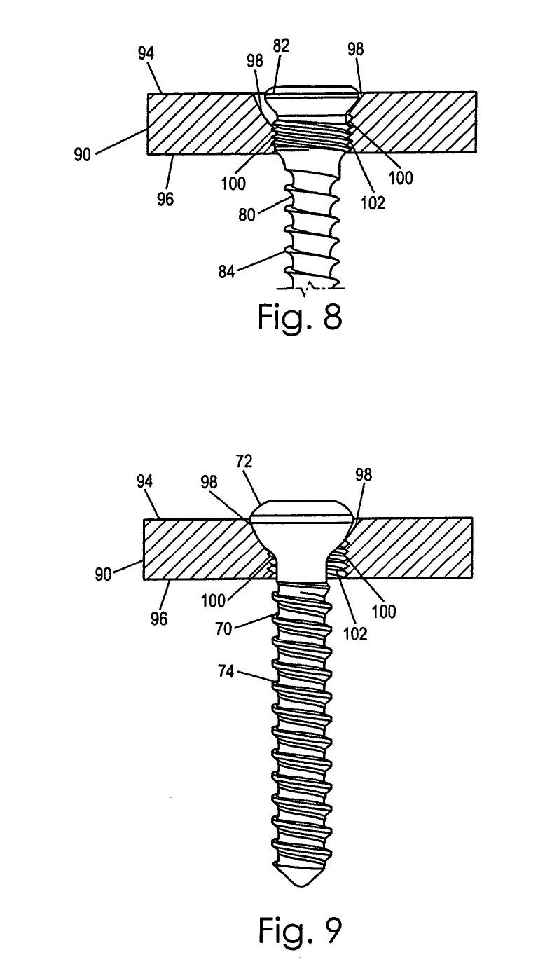

FIG. 8 shows a side view of locking screw 80 threaded into hole 92 of bone plate 90. Threads of head 92 of locking screw 90 are received by threads 102 of bone plate 90. Threads 102 completely surround the threads of head 92, and shaft 84 of locking screw 80 is threaded into bone (not shown). Head 82 of locking screw 80 is shaped such that its unthreaded portion bears against the ramp of top portion 98 of hole 92 of bone plate 90. Additionally, the threaded portion of head 82 is generally cylindrical (i.e., not tapered) so that it properly mates with threads 102 of hole 92 of bone plate 90. A threaded portion of a head of a locking screw for use with certain embodiments of this invention should be shaped to generally correspond to the shape of threaded portion of the hole of the bone plate.

FIG. 9 shows compression screw 70 inserted within hole 92 of bone plate 50. As shown in FIG. 9, head 72 of compression screw 70 sits within the concave recessed or spherical portion of top portion 98 of bone plate 90. Head 72 of compression screw 70 contacts the side of top portion 98 that includes the ramp, but head 72 does not completely abut the ramp of top portion 98. As shaft 74 is threaded into a bone (not shown), compression screw 70 may pull or push bone plate 90 in a particular direction as head 72 of compression screw 70 comes into contact with and rides along top portion 98 of hole 92 of bone plate 90, similar to that described above. In an exemplary embodiment, fine adjustment of fractures up to about two millimeters in several directions is possible. These figures show that locking and non-locking fixation elements may be used according to various embodiments of this invention.

Certain exemplary embodiments of bone plates according to this invention include holes, such as hole 52 or hole 92, that not only receive compression or locking screws interchangeably but also accept multiple types of compression screw heads with varying outer and inner diameters and thread forms. A compression screw can be placed through such holes and used for fixation, provided the minor diameter of the screw shank does not exceed the minor diameter of the hole. The diameter of the head of the compression screw should not be less than the minor diameter of the hole because the compression screw would not then rest on any part of the bone plate as is necessary for fracture reduction.

Any number of bone plates may have any of the hole configurations described. For example, holes 52 and 92 are capable of interchangeably receiving compression screws and locking screws. In addition to the holes described, however, bone plates may also include other openings configured to receive only locking screws or only compression screws, which is well understood by those skilled in the art. Bone plates may also include pinholes or provisional fixation holes or slots that may receive provisional fixation pins, which are also known in the art. There may also be provided non-circular openings in the plates that may or may not include threads depending on the purposes for which the openings are to be used.

In addition to the multi-functional holes described, bone plates 50 according to certain embodiments of the present invention also include a surgical cable clamping system. Various embodiments of clamps for use with this invention include a clamping mechanism on a bone plate, the clamping mechanism including a clamping body adapted to secure an orthopedic cable with respect to the bone plate and a securing member adapted to secure the clamping body to the bone plate. The clamping body is adapted to secure a first tension in the orthopedic cable, release the tension in the orthopedic cable, and re-secure the orthopedic cable relative to the clamping body to secure another tension in the orthopedic cable. The securing member is adapted to contact a portion of the clamping body, create a compression force on the portion of the orthopedic cable to secure the orthopedic cable relative to the clamping body with a first tension, release the compression force on the portion of the orthopedic cable so that the orthopedic cable can be released relative to the clamping body, and create a second compression force on the portion of the orthopedic cable to re-secure the orthopedic cable relative to the clamping body with a second tension.

A surgical cable clamp permits a surgeon to save time and reduce wastage during a surgical procedure by providing the option to reuse both a surgical cable clamp and orthopedic surgical cable that have been initially installed and tensioned. The surgeon may find that later during the same surgical procedure, the surgical cable clamp and orthopedic surgical cable should be retensioned, and the surgical cable clamp permits the surgeon to reclamp the orthopedic cable with respect to the installation of the bone plate.

As shown in FIG. 2, the preferred embodiment of the invention includes a surgical cable clamp 118 that is incorporated into bone plate 50 for securing the position of an orthopedic surgical cable relative to the bone plate and also relative to the bone to which the bone plate is to be attached.

As one skilled in the art will recognize, a surgical cable clamp can be fashioned as a single or multiple component-type clamp. In any configuration, a surgical cable clamp 118 is used to secure a tension and, if necessary, secure another tension in an orthopedic surgical cable without need for replacing the original surgical cable. The surgical cable clamps described herein can be used with the bone plate described above for securing the device to a patient's bone or another part of a patient's body. The clamp and bone plate combination provide greater flexibility in the operating room, e.g., it enables a surgeon to use a locking screw, a compression screw, or any other appropriate fixation device, such as a surgical cable, if needed or preferred.

The device-incorporated clamp 118 uses a portion of the bone plate 50 for clamping the orthopedic surgical cable. The bone plate 50 is adjacent to a patient's bone during an orthopedic surgical procedure. One or more orthopedic surgical cables can be utilized to secure the bone plate 50 into a position relative to the patient's bone. When a force is applied to a device-incorporated clamp 118, the device-incorporated clamp 118 compresses the orthopedic surgical cable, thus securing the orthopedic surgical cable into a position relative to the bone plate 50 and patient's bone.

If necessary, the orthopedic surgical cable can be loosened by applying another force to the device-incorporated clamp 118 to relieve the compression force on the orthopedic surgical cable applied by the device-incorporated clamp 118. The orthopedic surgical cable can then be retensioned by hand or by way of a tensioning device (not shown) so that the orthopedic surgical cable is at a desired tension or position. Yet another force can then be applied to the device-incorporated clamp 118 to create another compression force on the orthopedic surgical cable which can then maintain the desired tension or position of the orthopedic surgical cable. Depending upon the location of the orthopedic surgical cable relative to the bone plate 50 and the patient's bone or other bone, the device-incorporated clamp 118 may be used to secure the position and secure the tension of the orthopedic surgical cable.

FIG. 10 shows an embodiment of a bone plate 50 having an upper clamping body 122, an opening 126, and a securing member 128. The upper clamping body 122 has one or more cable receiving portions 132 that are adapted to secure a cable 140 between the clamp body 122 and the bone plate 50. In one embodiment, the cable receiving portion(s) 132 are a pair of semi-circular cable channels that are machined in the lower surface 210 of the clamping body 122. The cable channels are sized to receive the width of an orthopedic surgical cable 140 and are machined through the width of the upper clamping body 122 along the lower surface 210.

Through the upper surface 208, an opening 126 (which, in some embodiments, is a bolt hole for receiving a clamping bolt) is machined through the thickness of the clamping body 122 to the lower surface 210. Note that the upper clamping body 122 can have numerous other shapes and configurations in accordance with the invention. For instance, the upper clamping body 122 of FIG. 10 is shown as a rectangularly-shaped component that has a relatively flat profile. Other figures show clamping body 122 as having a generally rounded upper surface (see e.g. FIG. 12), a generally flat lower surface, a generally u-shaped or indented lower surface (see e.g. FIG. 13), or any other shape that can cooperate with a portion of bone plate.

As also shown in the Figures, securing member 128 is preferably inserted into the upper portion of bone plate.

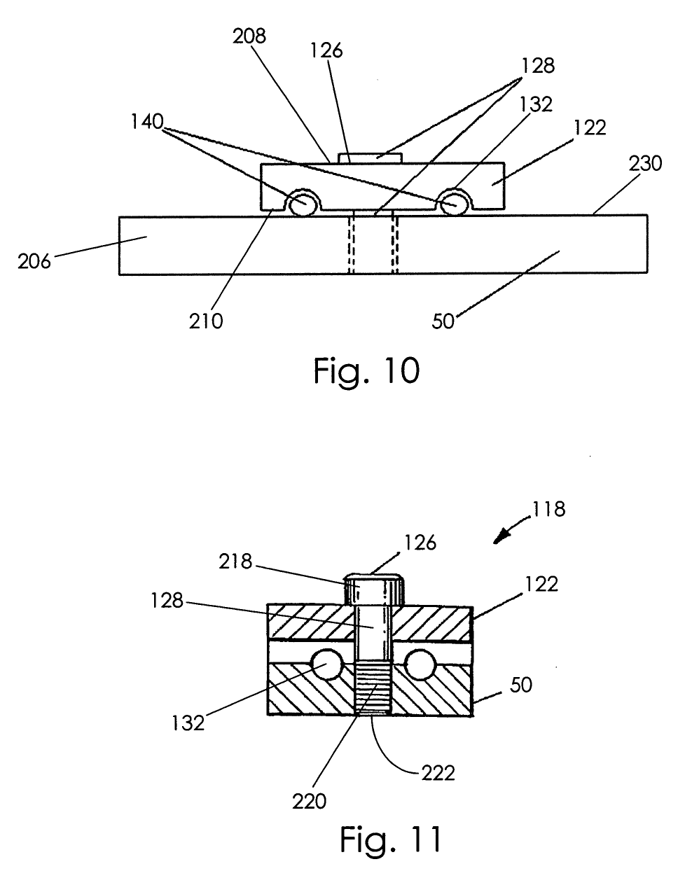

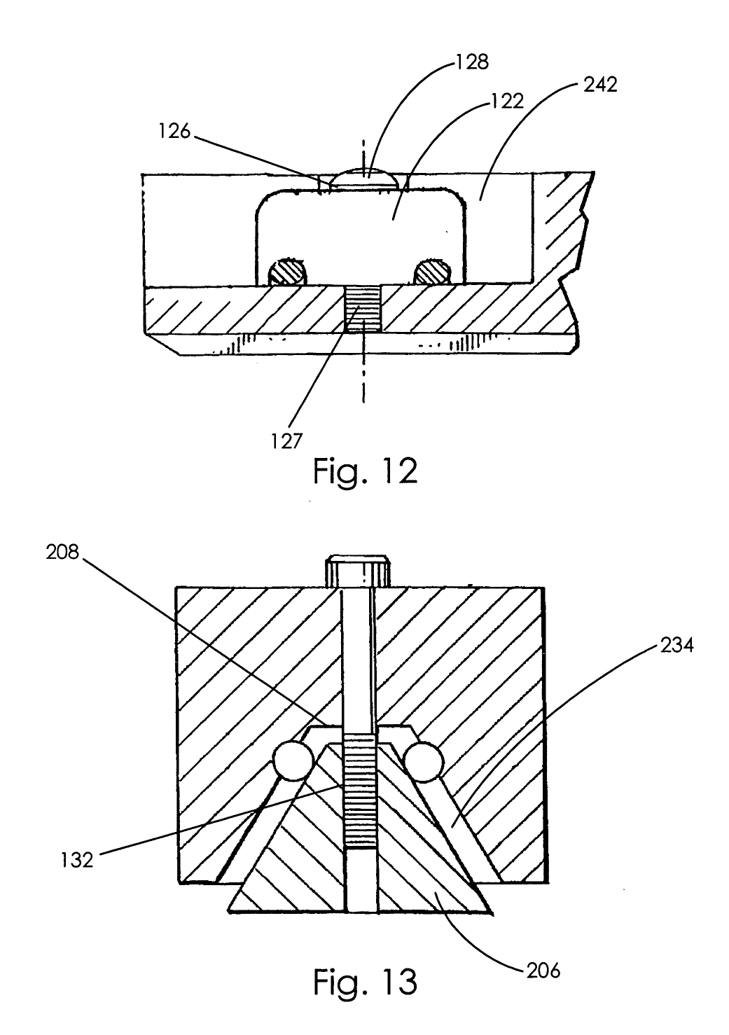

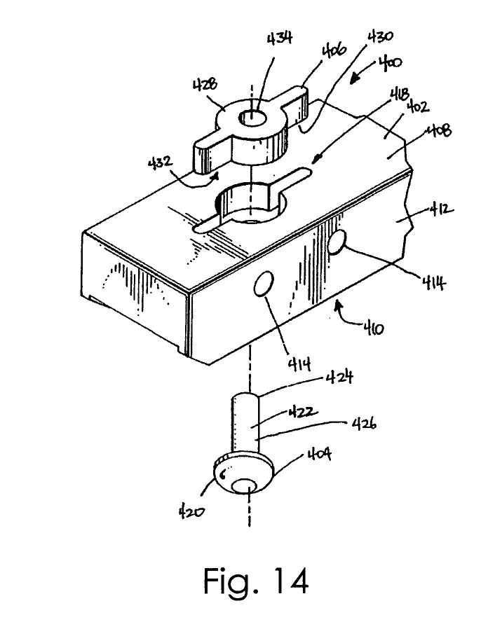

As shown in FIG. 11, securing member 128 may be shaped similar to a conventional machine screw with a socket head 218, a threaded body 220, and blunt point 222. The socket head 218 may include a recess sized to receive a hexagonal-shaped tightening instrument (not shown) for tightening and untightening the securing member 128 to a desired tension. Alternatively, the external shape of the socket head 218 can be shaped for tightening with a wrench-type instrument for tightening and untightening a corresponding geometrically-shaped socket head. The threaded body 220 is sized to diametrically fit within the opening 126 of the upper clamping body, and may include one or more threads sized to receive corresponding threads of threaded body 220. In some embodiments, the body is not fully threaded, but there is an unthreaded portion at the upper portion of the body near the socket head 218. This embodiment may allow the clamp to achieve greater compression. Note that the securing member 128 may have numerous other shapes and configurations in accordance with the invention.

The lower clamping body of clamp 118 is actually a part of bone plate 50. As shown in FIG. 10, it some embodiments, it has a generally flat upper surface 230 sized to receive the lower surface 210 of the upper clamping body 122. It may also feature a corresponding cable receiving portion 132 machined into its surface, as shown in FIG. 11. This part of bone plate may often be referred to as lower clamping body 206, although it is understood that in addition to serving as a part of clamp 119, it is also forms a portion of bone plate 50 itself.

In some embodiments, the cable receiving portion 132 includes a series of grooves or ridges machined in the length of the portion 132. A series of corresponding grooves or ridges may also be also machined in the length of the cable receiving portion 132 of the upper clamping body 122. This may help secure the cable in place.

An opening 127, preferably a threaded opening, is also machined through the thickness of the lower clamping body 206 of the bone plate 50 to the bone contacting surface 56. The lower clamping body (or in other words, the portion of the bone plate 50 that forms cable clamp) can have numerous other shapes and configurations in accordance with the invention. For example, as shown in FIG. 13, the body 206 may extend up to form a protrusion 132 and the lower surface 208 of the clamping body may form an indentation 234. Protrusion 232 and indentation 234 may be any appropriate shape that correspond to one another. (Additionally, protrusion 232 may be provided on clamping body 122 and indentation 234 may be provided on bone plate 50.) FIG. 13 shows a wedge shape, but it should be understood that the shapes may be triangular, round, oblong, trapezoidal, dove tail and slot, j-lock, or any other appropriate combination.

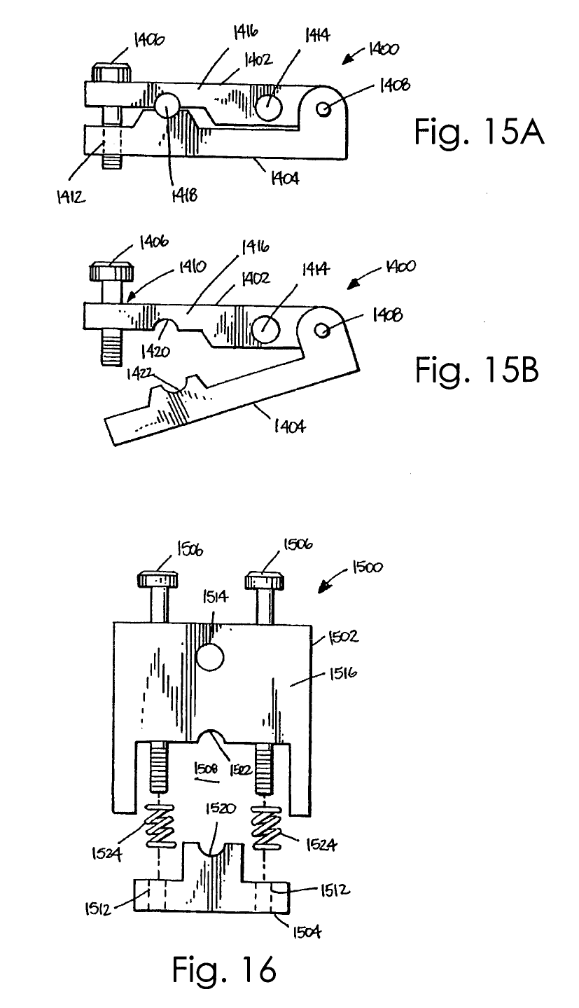

Upper clamping body 122 and lower clamping body portion of bone plate 50 may lay on one another as shown in FIG. 10 or may be received by one another, as shown in FIGS. 12 and 13. In other words, when securing member 128 is aligned with opening 126 of the upper clamping body 122 and opening 127 of lower clamping body 206, the ends of upper clamping body 122 may either lay on top of the lower clamping body, as shown in FIG. 11, or they may fit within recesses 242 of the lower clamping body, thus assisting alignment of the cable receiving portions 132, particularly if they are semi-circular-shaped cable channels of the upper clamping body 122 that cooperate with semi-circular-shaped cable channels of the lower clamping body 206 to form circular-shaped cable holes, as shown in FIGS. 15A, 15B and 16. In this configuration, the series of grooves (if provided) of the lower clamping body 206 and corresponding grooves (if provided) of the upper clamping body 122 align with each other to decrease the width of the circular hole formed by the alignment of the cable channels. Although various embodiments of configurations have been described, it is also important to note that one feature that is provided by the present invention is variability and options. Accordingly, it is possible for hole 52 of bone plate 50 to also function as opening 127 to receive clamp 118. This allows the use of a fixation element 40, 80 or 70 or a cable clamp 118 at the same location. In some embodiments, the clamp is attached to the bone plate through one of the holes in the plate. In other embodiments, it may be integrally formed with the bone plate. Alternatively, it may be wrapped around bone plate and secured to itself and either end, without being attached through one of the holes.

Hinge Option

FIGS. 15A and 15B show another embodiment of a surgical cable clamp. In this embodiment, a surgical cable clamp 1400 includes an upper clamping body 1402, a lower clamping body 1404 (which is actually a portion of bone plate), and a clamping bolt 1406. The upper clamping body 1402 is configured to hingably fit together with the lower clamping body 1404 via a hinge 1408. Together, the upper clamping body 1402 and lower clamping body 1404 form a V-shape. A bolt hole 1410 in the upper clamping body 1402 adjacent to an unhinged end corresponds with a threaded bolt hole 1412 in the lower clamping body 1404 adjacent to its unhinged end. Each of the bolt holes 1410, 1412 are sized to receive the clamping bolt 1406. The clamping bolt 1406 has a similar shape as the securing member shown and described above.

At least one cable hole 1414 is machined in a lateral side 1416 of the upper clamping body 1402. At an interface between the upper clamping body 1402 and lower clamping body 1404, a second cable hole 1418 is formed when the upper clamping body 1402 fits together with the lower clamping body 1404. For example, a recessed portion 1420 of the upper clamping body 1402 can be a concave-shaped cable channel, and a recessed portion 1422 of the lower clamping body 1404 can be a concave-shaped cable channel that corresponds to the recessed portion 1420 of the upper clamping body 1402 to form a second cable hole 1418. The cable hole 1410 and second cable hole 1418 are sized to receive an orthopedic surgical cable (not shown) to be clamped and reclamped by the surgical cable clamp 1400.

When an orthopedic surgical cable is inserted within either or both the cable hole 1410 and second cable hole 1418, the upper clamping body 1402 can then be secured together with the lower clamping body 1404 by the clamping bolt 1406. The compression force of the upper clamping body 1402 upon the surgical cable secures the position of the cable relative to the lower clamping body 1404. By tightening and untightening the clamping bolt 1406, the surgical cable clamp 1400 can clamp and unclamp the orthopedic surgical cable as needed when tensioning the orthopedic surgical cable as desired. A series of grooves (not shown) or ridges to increase the friction or grip on the surgical cable can be machined within the second cable hole 1418 by machining the upper clamping body 1402 and/or lower clamping body 1404.

Spring Option

FIG. 16 illustrates another embodiment of a surgical cable clamp in accordance with the invention. FIG. 16 is an exploded side view of this embodiment of a surgical cable clamp in an unclamped position. In this embodiment, a surgical cable clamp 1500 includes an upper clamping body 1502, a lower clamping body 1504 (which is again, actually a portion of bone plate 50), and a pair of clamping bolts 1506. The lower clamping body 1504 forms an inverted T-shape and integrally fits within a corresponding recess 1508 in the lower portion of the upper clamping body 1502. The clamping bolts 1506 fit within a pair of respective bolt holes 1510 machined through portions of the upper clamping body 1502 and within corresponding threaded bolt holes 1512 machined in the lower clamping body 1504. Note that the clamping bolts 1506 each have a similar shape as the clamping bolt shown and described in FIG. 6. At least one cable hole 1514 is machined in a lateral side 1516 of the upper clamping body 1502. A second cable hole 1518 is formed when the upper clamping body 1502 is fit together with the lower clamping body 1504. For example, a tip portion 1520 of the T-shaped lower clamping body 1504 can have a concave-shaped tip and a corresponding recessed portion 1522 in the upper clamping body 1502 can be a concave-shaped portion that forms a second cable hole 1518 when the upper clamping body 1502 is integrally fit together with the lower clamping body 1504. The cable hole 1510 and second cable hole 1518 are sized to receive an orthopedic surgical cable (not shown) to be clamped and reclamped by the surgical cable clamp 1500.

One or more springs 1524 may be positioned between the upper clamping body 1502 and the lower clamping body 1504 to assist with the disassembly of the upper clamping body 1502 from the lower clamping body 1504. In the example shown, the springs 1524 are concentrically positioned around the clamping bolts 1506, and are configured to compress when the lower clamping body 1502 is compressed within the recess 1508 of the upper clamping body.

When an orthopedic surgical cable is inserted within either or both the cable hole 1510 and second cable hole 1518, the lower clamping body 1504 can then be fit together with the upper clamping body 1502, and then the lower clamping body 1504 is secured to the upper clamping body 1502 by the clamping bolts 1506. The compression force of the lower clamping body 1504 upon the surgical cable secures the position of the cable relative to the upper clamping body 1502. By tightening and untightening either or both of the clamping bolts 1506, the surgical cable clamp 1500 can clamp and unclamp the orthopedic surgical cable as needed when tensioning the orthopedic surgical cable as desired. A series of grooves (not shown) or ridges to increase the friction or grip on the surgical cable can be machined within the second cable hole 1518 by machining the upper clamping body 1502 and/or lower clamping body 1504.

The plate and clamp combination may be manufactured from titanium, stainless steel, cobalt chromium alloy, or another similar type of material. An example of a clamping bolt is a conventional #8 machine screw made from titanium, stainless steel, cobalt chromium alloy, or a similar type of material that is compatible with material of the upper and lower clamping body. In some instances, the clamping bolt may be coated with an implantable coating designed to reduce frictional contact with other components of the clamp. Furthermore, an example of a surgical cable that can be used with the stand alone-type clamp 200 is typically a cobalt chromium or stainless steel cable measuring approximately 0.04 to 0.08 inches (1.0 to 2.0 mm) in diameter.

Use of Clamps:

Bone plate 50 is aligned with a proximal end of a patient's femur bone in accordance with a hip replacement procedure. When the bone plate 50 is to be secured to the patient's femur, a predetermined length of surgical cable is inserted into and pulled through a cable receiving portion 132 of the clamp 118. There may be provided a bead on a relatively larger diameter end of the surgical cable that secures the relatively larger diameter end of surgical cable adjacent to the surgical cable clamp when the length of the surgical cable is pulled through the first cable hole. This method and the bead is described more fully in co-pending Application Publication Number 2004/0087954.

The cable is wrapped around the thickness of the patient's femur and inserted through a second cable receiving portion 132 of the surgical cable clamp 118. This may be accomplished using a cable tensioning device until a desired tension is attained. When the surgical cable is pulled to a desired tension, the securing member 128 is placed and tightened (e.g., with a hexagonal-shaped tightening instrument or a T-handled driver) until a compression force between the upper clamping body 122 and the recess (lower clamping body 200 of bone plate 50) maintains the desired tension on the surgical cable. Any excess length of surgical cable may be trimmed with a cutting instrument.

(A suitable cable tensioning device can be a device or system that applies a tension to a surgical cable, maintains the tension on the surgical cable until the tightening instrument can be used to tighten the securing member (e.g., clamping bolt of the surgical cable clamp), measures the tension in the surgical cable, and releases the surgical cable when the clamping bolt has secured the surgical cable.)

More than one surgical cable may be needed to secure a bone plate. Accordingly, multiple clamps may be provided on the plate and the above sequence can be repeated as needed until the bone plate is secured to the patient's femur or bone. After tensioning one or more surgical cables to the patient's femur with one or more corresponding surgical cable clamps 118, previously tensioned surgical cables may tend to loosen or otherwise require additional tension to sufficiently secure the bone plate to the patient's femur. If necessary, the tension on a previously tensioned surgical cable can be released by applying an untightening force to the securing member 128, releasing the compression force between the upper clamping body 122 and lower clamping body 206, thus releasing the compression and tension on the surgical cable. The surgical cable is then retensioned manually or by use of the cable tensioning device. When the desired tension is reached, a tightening force is applied to the securing member in order to create a sufficient compression force between the upper clamping body and the lower clamping body to maintain the desired tension in the surgical cable 306, and secure the position of the surgical cable relative to the surgical cable clamp.

If necessary, the orthopedic surgical cable can be loosened by applying another force to the device-incorporated clamp 118 to relieve the compression force on the orthopedic surgical cable applied by the device-incorporated clamp 118. The orthopedic surgical cable can then be retensioned by hand or by way of a tensioning device so that the orthopedic surgical cable is at a desired tension or position. Yet another force can then be applied to the device-incorporated clamp 118 to create another compression force on the orthopedic surgical cable which can then maintain the desired tension or position of the orthopedic surgical cable. Depending upon the location of the orthopedic surgical cable relative to the bone plate 50 and the patient's femur bone or other bone, the device-incorporated clamp 118 may be used to secure the position and secure the tension of the orthopedic surgical cable.

Changes and modifications, additions and deletions may be made to the structures and methods recited above and shown in the drawings without departing from the scope or spirit of the invention and the following claims.

What is claimed is:

1. A bone plate and cable clamp system, comprising:
a bone plate having an upper surface, a bone contacting surface, and a plurality of holes, the plurality of holes extending through the bone plate between the upper surface and the bone contacting surface;
at least one of the holes adapted to interchangeably receive a locking fixation element or a compression fixation element, the at least one hole having a thread that makes a complete revolution around the hole;
a clamping mechanism adapted to be attached to the plate, the clamping mechanism comprising
(a) a clamping body,
(b) a securing member, and
(c) a cable receiving portion in or on the clamping body or in or on the bone plate,
the clamping mechanism adapted to be secured to the bone plate such that a surgical cable can be received by the cable receiving portion and secured in one position, released if necessary, and re-secured in the same or different position without causing damage to the cable.

2. The system of claim 1, wherein the clamping mechanism (1) receives a surgical cable and creates a compression force on a portion of the cable to secure the cable relative to the clamping mechanism with a first tension, (2) releases the compression force on the portion of the cable so that the cable can be released relative to the clamping body, and (3) creates a second compression force on the portion of the cable to re-secure the cable relative to the clamping mechanism with a second tension.

3. The system of claim 1, wherein the clamping body has an opening and wherein the securing member is received in the opening.

4. The system of claim 1, wherein the securing member comprises a clamping bolt adapted to mount in a corresponding hole of the clamping body.

5. The system of claim 1, wherein the cable receiving portion comprises a grooved channel to receive a portion of the cable.

6. The system of claim 1, wherein the cable receiving portion comprises an opening that extends through the clamping body.

7. The system of claim 1, wherein the cable receiving portion comprises an opening in the bone plate.

8. The system of claim 1, wherein the cable receiving portion comprises at least a partial opening in the clamping body and at least a partial opening in the bone plate.

9. The system of claim 1, wherein the plurality of holes are adapted to receive one or more of a locking element, a compression element, or a clamping mechanism.

10. The system of claim 1, where at least one of the holes comprises an upper portion and a lower portion.

11. The system of claim 1, wherein the clamping mechanism is attached to the plate through one of the plurality of holes in the plate.

12. The system of claim 1, wherein the clamping mechanism is integrally formed with the plate.

13. The system of claim 1, wherein the clamping mechanism is attached to the plate by being wrapped around the plate.

14. The system of claim 1, wherein one of the plurality of holes receives a fixation element in use and wherein one of the plurality of holes receives a clamping mechanism in use.

15. A method for using a combination bone plate and cable clamp system, comprising:
(a) providing a bone plate having an upper surface, a bone contacting surface, and a plurality of holes, the plurality of holes extending through the bone plate between the upper surface and the bone contacting surface, at least one of the holes adapted to interchangeably receive a locking fixation element or a compression fixation element, the at least one hole having a thread that makes a complete revolution around the hole;
(b) providing a clamping mechanism adapted to be attached to the plate, the clamping mechanism comprising (i) a clamping body, (ii) a securing member, and (iii) a cable receiving portion in or on the clamping body or in or on the bone plate,
(c) inserting a fixation element into one of the plurality of holes; and
(d) inserting a surgical cable into the clamping mechanism.

16. The method of claim 15, wherein the clamping mechanism is inserted into one of the plurality of holes.

17. A bone plate and cable clamp kit, comprising:
(a) a bone plate having an upper surface, a bone contacting surface, and a plurality of holes, the plurality of holes extending through the bone plate between the upper surface and the bone contacting surface, at least one of the holes adapted to interchangeably receive a locking fixation element or a compression fixation element, the at least one hole having a thread that makes a complete revolution around the hole;
(b) a clamping mechanism adapted to be attached to the plate, the clamping mechanism comprising (i) a clamping body, (ii) a securing member, and (iii) a cable receiving portion in or on the clamping body or in or on the bone plate, the clamping mechanism adapted to be secured to the bone plate such that a surgical cable can be received by the cable receiving portion and secured in one position, released if necessary, and re-secured in the same or different position without causing damage to the cable.
(c) one or more fixation elements to be received by the bone plate; and
(d) one or more surgical cables to be received by the clamping mechanism.
